(19) United States
(12) Patent Application Publication
(10) Pub. No.: US 2021/0196176 A1
(43) Pub. Date: Jul. 1, 2021

(54) DEFECATORY DISORDER ASSESSMENT METHODS

(71) Applicant: Mayo Foundation for Medical Education and Research, Rochester, MN (US)

(72) Inventor: Adril E. Bharucha, Rochester, MN (US)

(21) Appl. No.: 17/131,490

(22) Filed: Dec. 22, 2020

Related U.S. Application Data
(60) Provisional application No. 62/954,251, filed on Dec. 27, 2019.

(51) Int. Cl. A61B 5/22; A61B 5/00

(52) U.S. Cl. CPC A61B 5/227; A61B 2562/0247; A61B 5/0053; A61B 5/6853

(57) ABSTRACT

This document describes methods for assessing defecatory disorders of patients. For example, this document describes methods for assessing evacuation using seated manometry and methods for analyzing rectoanal pressures to diagnose pelvic floor dysfunction.

### CROSS-REFERENCE TO RELATED APPLICATIONS

This application claims the benefit of U.S. Provisional Application Ser. No. 62/954,251, filed Dec. 27, 2019. The disclosure of the prior application is considered part of (and is incorporated by reference in) the disclosure of this application.

### BACKGROUND

### 1. Technical Field

This document relates to methods for assessing anorectal functions and diagnosing defecatory disorders in patients. For example, this document relates to methods for assessing evacuation using seated manometry and to methods for analyzing rectoanal pressures to diagnose pelvic floor dysfunction.

### 2. Background Information

Defecatory disorders are defined by symptoms of constipation and objective evidence of impaired rectal evacuation. Impaired evacuation may result from increased resistance to evacuation and/or inadequate rectal propulsive forces. High resting anal pressure, incomplete relaxation, or paradoxical contraction of the puborectalis and external anal sphincters (dyssynergia) cause increased resistance to evacuation.

The median prevalence of constipation is 16% in all adults. In older people, the prevalence is greater (i.e., 33.5% in adults aged 60-101 years). Factors such as increasing age, female gender, lower socioeconomic status, lower parental education rates, less self-reported physical activity, certain medications, stressful life events, physical and sexual abuse, and depression are associated with constipation.

Anorectal manometry is an examination of the muscle function in the anorectal area. It consists of placing into the rectum a flexible tube with multiple sensors that detect pressure in the rectum and anal canal.

### SUMMARY

This document describes methods for using manometry to diagnose defecatory disorders of patients. For example, this document describes methods for assessing evacuation using seated manometry and methods for analyzing rectoanal pressures to diagnose pelvic floor dysfunction.

In one implementation, a method for assessing defecatory characteristics of a patient includes performing high-resolution anorectal manometry on the patient while the patient is in a seated position.

Such a method for assessing defecatory characteristics of a patient may optionally include one or more of the following features. The method may further comprise analyzing rectoanal pressure results from the high-resolution anorectal manometry. The analysis may include classifying a rectoanal pressure profile of the patient from the high-resolution anorectal manometry into one of four groups: (i) minimal change, (ii) anal relaxation, (iii) paradoxical contraction, and (iv) transmission. The method may also include analyzing rectoanal pressure results from the high-resolution anorectal manometry. The analyzing may include determining a weighted combination of: (i) anal resting pressure, (ii) rectal and anal pressures and (iii) rectoanal gradient during evacuation. The performing high-resolution anorectal manometry may include using a catheter comprising two rectal balloons with pressure sensors and ten additional pressure sensors spaced apart from each other along the catheter. In some embodiments, at least seven pressure sensors of the ten additional pressure sensors are in the patient's anal canal while performing the high-resolution anorectal manometry.

Particular embodiments of the subject matter described in this document can be implemented to realize one or more of the following advantages. The methods described herein for assessment of evacuation using seated manometry are a substantial improvement over conventional methods that use left lateral manometry. Accordingly, the methods described herein provide improved techniques for diagnosing pelvic floor dysfunction, such as in constipated patients. In addition, this disclosure describes new methods for analyzing and classifying rectoanal pressure patterns that provide enhanced clinical analytical techniques.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described herein. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description herein. Other features, objects, and advantages of the invention will be apparent from the description and drawings, and from the claims.

### DESCRIPTION OF THE DRAWINGS

FIG. 1 is a flowchart describing a new method for analyzing and classifying rectoanal pressure patterns into four rectoanal pressure profiles in accordance with some embodiments provided herein.

FIG. 2 provides plots of measured rectal and anal pressures the four rectoanal pressure profiles at rest and during evacuation. The plots provide median and inter-quartile rectal and anal pressures for each category.

FIG. 3 is a chart showing a comparison of pressure patterns in the left lateral and seated positions in participants. Participants on the perimeter of the circle had the same pattern in both positions and a normal balloon expulsion test. Participants inside the circle had a different pattern in left lateral and seated positions and a normal balloon expulsion test inside. Participants outside the circle had an abnormal balloon expulsion test. In the seated position, nearly 50% of participants had the minimal change pattern.

FIG. 4 illustrates three receiver operating characteristic curves showing the utility of anal manometry features for discriminating between constipated patients without and with a defecatory disorder in the left lateral position (Panel A), seated position (Panel B), and left lateral versus seated positions (Panel C).

FIG. 5 is a flow chart depicting a method that uses the new analysis for diagnosing defecatory disorders in the upright position. Among patients with transmission or anal relaxation documented with manometry, the risk of a defecatory disorder is low; a normal BET effectively excludes a defecatory disorder. In the other patterns, the rectoanal gradient is useful for estimating the likelihood of a defecatory disorders and guiding further tests. This method can be used in the left lateral position by substituting upright values with left lateral values of the rectoanal gradient.

Like reference numbers represent corresponding parts throughout.

### DETAILED DESCRIPTION

This document describes methods for assessing defecatory disorders (DD) of patients. For example, this document describes methods for assessing evacuation using seated manometry and methods for analyzing rectoanal pressures to diagnose pelvic floor dysfunction.

Conducted in the left lateral position, anorectal manometry, whether conducted with conventional (non-high resolution manometry) or high resolution manometry (HRM), is inadequate for diagnosing DD because findings overlap considerably with healthy people. This disclosure provides techniques for improving the diagnostic utility of manometry by evaluating pressures in the seated position and analyzing rectoanal pressures with a novel approach.

As described further herein, the assessment of evacuation using seated manometry and the new techniques for analysis of rectoanal pressures provide techniques that are superior to conventional left lateral manometry for diagnosing pelvic floor dysfunction in constipated patients.

Among constipated patients, anorectal tests, beginning with anorectal manometry accompanied with a rectal balloon expulsion test (BET) and/or barium or magnetic resonance defecography, are conventionally used to diagnose DD. Different anorectal tests often provide different answers among individual patients. Manometry is generally the initial and arguably the most widely test used to diagnose DD.

However, recent studies have concluded that anal manometry is of limited utility for diagnosing DD because, even in asymptomatic people, the rectoanal pressure gradient (i.e., rectal pressure minus anal pressure) during evacuation is negative, and overlaps considerably with DD patients. Even a more refined approach in which rectoanal pressures are used to characterize patterns (e.g., reduced rectal propulsive forces, impaired anal relaxation, and both [i.e., hybrid pattern]) may fail distinguish between healthy controls and DD patients.

During evacuation, which typically lasts 20 seconds, widely used HRM software acquires data at 10 Hz and identifies the 3 second period over which the rectoanal gradient is highest, i.e., most conducive to evacuation. Highest in this context refers to the highest average over any 3 second period during the 20 second epoch. For each of the 30 samples in this 3 second average gradient, the anal pressure is actually the highest pressure at any level of the anal canal. Because the rectoanal gradient is derived by subtracting anal from rectal pressure, the gradient, may not be the highest but actually the lowest gradient at that instant. Perhaps this explains why the rectoanal gradient during evacuation is negative in most asymptomatic people, which limits the utility of HRM for diagnosing DD. Only one of several (e.g., up to ten) anal sensors is used to calculate the gradient. This sensor is individualized per study, hence varies among patients. Finally, the analysis of anorectal HRM, which is exclusively based on numerical measurements, has lagged esophageal HRM, wherein the pressure topography underpins a classification of disorders (e.g., subtypes of achalasia).

In view of the deficiencies of the conventional DD diagnostic techniques, the inventors postulated that the utility of HRM is limited because HRM is performed in the left lateral position and/or because conventional methods for summarizing rectoanal pressures during evacuation are suboptimal. In seeking to address these limitations, the aims of this study were to compare rectoanal pressures measured in the left lateral and seated positions, the utility of left lateral and seated FIRM, and the analysis of rectoanal pressure patterns with the existing and a new approach for discriminating between healthy controls, constipated patients without DD, and constipated patients with DD.

### Experimental Methods

After administering two sodium phosphate enemas, rectal BET, left lateral HRM, and seated HRM were measured at rest, during squeeze, and during simulated evacuation in 64 healthy women and 136 constipated women. The data was analyzed by conventional methods and by new methods for discriminating between constipated patients with normal and abnormal expulsion time (i.e., DD).

Anorectal Manometry

Recto-anal pressures were measured with Manoscan™ (Medtronic Inc, Minneapolis, Minn., USA) high-resolution manometry catheters. Of the catheter's pressure sensors, two are in the rectal balloon and ten, spaced at 6 mm intervals, are positioned in the anal canal. Pressures were measured for 20 seconds at rest, during squeeze (voluntary contraction of the anal sphincter, 3 attempts), simulated evacuation with an empty rectal balloon, and a Valsalva maneuver. Rectal sensory thresholds for first sensation, urge and discomfort were recorded while the balloon was progressively distended in 20 ml increments from 0 to 200 ml and, if necessary thereafter, in 40 ml increments until 400 ml. This procedure was performed in the left lateral and subsequently seated positions while seated on the commode. During seated HRM, the manometry catheter was externally enclosed within a plastic clip and attached to the inner thigh with an adhesive tape.

All results of the studies were analyzed with the latest version of commercially-available software (Manoview™ AR v3.0; Medtronic Inc), and also with a new method as described herein. Differences between these methods are described below in Table A, and elsewhere herein. In contrast to the Manoview™ analysis, the new analysis method classifies rectoanal pressure patterns into four types, and interprets these rectoanal pressures in the context of the underlying pattern.

TABLE A
Definitions of Rectoanal Parameters Evaluated with Manoview ™ and
with the New Analysis Technique

| | | New Analysis |
|---|---|---|
| | Manoview ™ | Method |
| Rectal pressure - rest | Difference between | Highest instantaneous |
| | anal pressure absolute | pressure averaged |
| | reference and anal | over 1.5 seconds |
| | pressure rectal | before evacuation |
| | reference, during | |
| | rest maneuver | |
| Anal pressure rest | Average of the | Average anal |
| | maximum anal | pressures in channels |
| | pressure across | 8, 9, 10 from 0-1.5 |
| | the anal canal | seconds in the |
| | (eSleeve) for 20 | evacuation maneuver |
| | seconds | |
| Rectal pressure - | Rectal pressures | Highest instantaneous |
| evacuation | during the 3 second | pressure in channels |
| | epoch when RAG | 1 and 2 averaged over |
| | is least negative or | during 5-15 seconds |
| | most positive | of evacuation |
| Anal pressure - | Average of the | Average of anal |
| evacuation | maximum anal | pressures in channels |
| | pressure during | 8, 9, 10 during 5-15 |
| | the 3 second epoch | seconds of evacuation |
| | when RAG is | |
| | least negative or | |
| | most positive | |
| Rectal pressure | Rectal pressure | Rectal pressure change |
| increment | change | (evacuation - rest) |
| | (evacuation - rest) | |
| Change in anal pressure | Anal pressure | Anal pressure change |
| during evacuation | change | (evacuation - rest) |
| | (evacuation - rest) | |
| Rectoanal gradient | (Rectal - anal | (Rectal - anal pressure) |
| during evacuation | pressure) during | during evacuation |
| | evacuation | |

The present disclosure pertains in part to new uses for a HRM catheter which comprises two rectal balloon sensors that are 3.4 cm proximal to the uppermost of 10 sensors, numbered 3-12 herein, which are separated by 0.6 mm (i.e., they span 5.4 cm). The most distal sensors, i.e., 12, and often 11, are outside the body. While sensors 7-10 are generally located in the anal canal, and sensors 3 and 4 are typically in the rectum, sensors 5 and 6 may be located either in the rectum or anal canal, depending on the anal canal length.

For the analysis, sensors that recorded a baseline pressure greater than 30 mm Hg were considered to be in the functional anal canal. The rectoanal pressures were averaged over 1.5 seconds immediately before evacuation, and between 5 and 15 seconds after the onset of the evacuation maneuver. These averages were analyzed. Pressures recorded during 0 to 4.9 seconds of evacuation were not utilized because anal pressures often increase at the outset of evacuation even in healthy people.

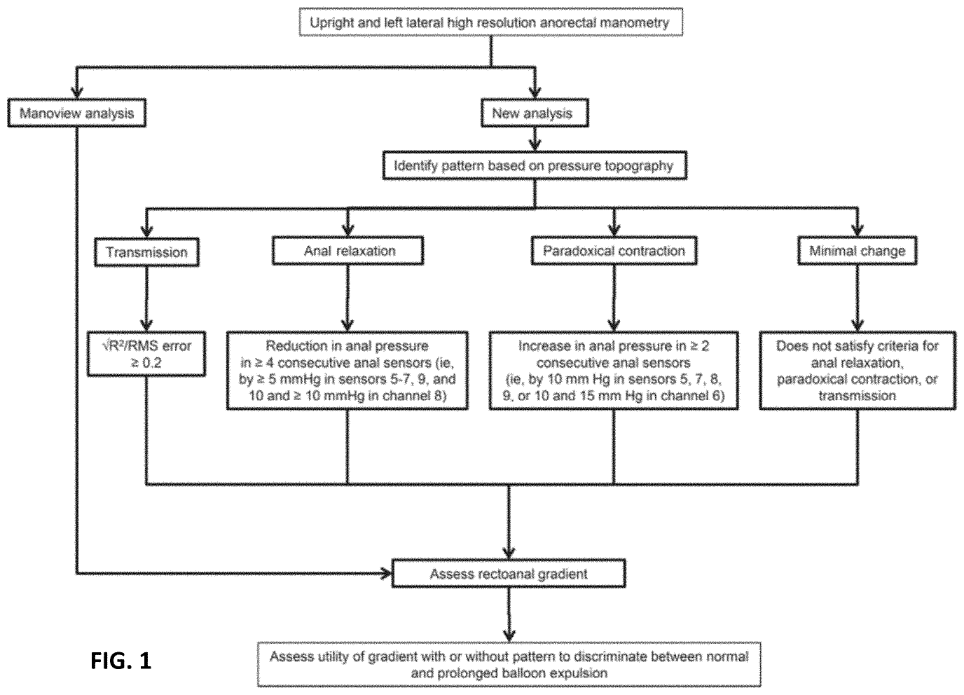

The difference (evacuation−baseline) was calculated for rectal and individually for all anal sensors as defined above. Based on the change (i.e. evacuation−rest) in anal pressures all HRM tracings in healthy people and patients, four patterns were identified (classified into four categories) as shown in FIG. 1. The four categories are: (i) minimal change, (ii) anal relaxation, (iii) paradoxical contraction, and (iv) transmission.

“Anal relaxation” was defined by sufficient anal relaxation during evacuation. To be conservative, anal relaxation was defined by reduction in anal pressures that exceeded a threshold in four or more consecutive anal pressure sensors. The thresholds for relaxation were 5 mmHg or greater in sensors 5-7, 9, and 10 and 10 mmHg in sensor 8.

Conversely, “paradoxical contraction” was defined by an increase in anal pressure in two or more consecutive sensors. The threshold change was 10 mm Hg in sensors 5, 7, 8, 9, and 10 and 15 mm Hg in sensor 6. Guiding the selection of these thresholds, approximately one-third (i.e., sensors 5, 6, and 10) or two-thirds (i.e., sensors 7-9) of healthy people with an nBET had anal relaxation as defined by these criteria (unpublished data). Conversely, only approximately 10% of healthy people had paradoxical contraction. Participants who had anal relaxation in some sensors and paradoxical contraction in other sensors were included in the latter category because anal contraction impedes evacuation. Those who did not have criteria for either pattern were defined as “minimal change.”

“Transmission,” which is visually evident, was defined by transmission of rectal pressures throughout the anal canal, suggestive of a common cavity. To identify this pattern, the linear regression of rectal and anal pressures during evacuation over the length of the anal canal (sensors 3 to 10) were analyzed in each patient. Two parameters in this regression, the square root of R,² which is the slope, and the root mean square (RMS) error, which represents the closeness of fit between the observed and the fitted data, were used to define transmission. When pressures decline from the rectum (sensor 3) to the distal anal canal (sensor 10), the √R2 is high. A low root mean square (RMS) error suggests a close fit between the observed and the fitted data. A √R2/RMS error≥2 was used to define transmission.

With suitable modification, similar criteria can also be applied to classifying patterns identified with other manometry catheters.

Rectal Balloon Expulsion Time

Participants had up to 3 minutes to expel a 4-cm-long balloon filled with 50 ml water from the rectum in privacy while seated on a commode. The balloon expulsion time was noted and the balloon was removed if participants could not expel the balloon within 3 minutes.

Statistical Analysis

The paired and unpaired t tests were used to compare the outcome variables (rectal and anal pressures and the RAG) between the LL and seated positions in healthy people and constipated patients and between constipated patients with a nBET and a prolonged BET (pBET). For each abnormal rectoanal parameter, likelihood ratios (LR) were used to express the likelihood (sensitivity/[1−specificity]) of a DD relative to constipation without DD, i.e., in patients with constipation with pBET versus constipation with nBET. The incremental probability was calculated as follows: 0.19×log likelihood ratio.

Among constipated patients, the pre-test and, based on the likelihood ratios, the post-test probabilities of having disease were computed. Categorical variables were compared with the chi-square test. Receiver operating characteristic (ROC) curves that were derived from logistic regression models were used to compare the utility of the RAG computed with the ManoView™ analysis and the new analysis and the addition of rectoanal pressure patterns, assessed in the LL and seated positions, for discriminating between constipated patients with nBET and pBET. Similar comparisons, albeit incorporating the BET as a continuous rather than a dichotomized variable, were performed with univariate and multiple Cox proportional regression models. Unless stated otherwise, the data are summarized as the mean (SD). All analyses used JMP software (version 9.4, SAS Cary, N.C.). Comparison between models used the model partial chi-square statistics in the case of nested models and used “DeLong and Delong” in the case of non-nested models.

As appropriate, parametric and non-parametric tests (i.e., t-test and Wilcoxon signed rank test) were used to compare the outcome variables (i.e., rectal and anal pressures and the rectoanal gradient) between the left lateral and seated positions, healthy patients and constipated patients with an abnormal BET, and separately constipated patients with a normal and an abnormal BET. Categorical variables were compared with the Fischer's exact test. ROC curves that were derived from logistic regression models were used to compare the utility of the rectoanal gradient assessed in the left lateral and seated positions for discriminating between healthy women with a normal BET (<60 sec) (the reference group), DD with normal BET, and DD with a prolonged BET. The data are summarized as the median value and inter-quartile ranges.

### Results

Among patients, the expulsion time was normal in 84 and prolonged in 52 patients (indicating a DD). In all participants, rectal and anal pressures and the rectoanal gradient during evacuation were greater in the seated than the left lateral position. With the Manoview™ analysis, the seated rectoanal gradient more effectively discriminated between constipated patients with normal and abnormal BET than the left lateral gradient (positive likelihood ratio=4.8 versus 3.1). Differences in receiver operating characteristic curve AUC of 0.80 vs 0.71) were significant (p=0.02).

Demographic Features and Clinical Characteristics

The age and BMI were not significantly different among controls (35±13y, 26±5 kg/m²), constipated patients with a normal BET (42±17y, 25±5 kg/m²) and constipated patients with an abnormal BET (41±14y, 24±5 kg/m²). Among 84 patients with normal BET, 38 (45%) had symptoms of functional constipation and 40 (48%) had symptoms of constipation-predominant IBS. Among 52 patients with a BET longer than 60 seconds, 32 (62%) had symptoms of functional constipation and 17 (33%) had constipation-predominant IBS; the BET was longer than 180 seconds in 44 of 52 (85%) of participants.

Anorectal Pressures Analyzed with Manoview™ Software in the Left Lateral and Seated Positions

With a few exceptions, rectal and anal pressures at rest and during evacuation were generally greater in the seated than the left lateral position in healthy controls and constipated patients (see Tables 1A-1C below). The most striking differences were for rectal pressure at rest (i.e., for controls; 4±7 mmHg in left lateral (LL) and 32±20 mmHg in seated position (UP), p<0.0001, for patients; 6±8 mmHg in LL and 28±16 mmHg in seated position, p<0.0001) and during simulated evacuation (i.e., for controls; 33±18 mmHg in LL and 63±34 mmHg in seated position, p<0.0001, for patients; 33±28 mmHg in LL and 62±36 mmHg in seated position, p<0.0001). To a lesser extent, the anal pressures at rest (i.e., for controls; 85±24 mmHg in LL and 97±27 mmHg in seated position, p=0.0003, for patients; 90±28 mmHg in LL and 105±34 mmHg in seated position, p<0.0001) and during simulated evacuation (i.e., for controls; 73±22 mmHg in LL and 83±25 mmHg in seated position, p=0.0006, for patients; 83±27 mmHg in LL and 97±34 mmHg in seated position, p<0.0001) were also greater in the seated than the LL position. Hence, the rectoanal gradient was significantly greater (i.e., less negative) in the seated than the LL position in controls and in patients.

Anal pressures during squeeze were lower in seated position than left lateral position in healthy women (P=0.01) and constipated patients (P<0.0001). Of the sensory thresholds, only the threshold for the first sensation was higher (P=0.02) in the seated (UP) than the LL position.

TABLE 1A

| | Healthy (n = 64) | | |
|---|---|---|---|
| | Left | | |
| | lateral | Seated | p- |
| Parameter | (LL) | (UP) | value |
| Anal pressure - rest, Manoview | 85 ± 24 | 97 ± 27 | .0003 |
| Anal pressure - rest, new analysis | 67 ± 26 | 78 ± 28 | .0001 |
| Anal pressure - squeeze, Manoview | 203 ± 58 | 192 ± 52 | .01 |
| Anal pressure - squeeze, new analysis | | | |
| Anal pressure - Valsalva, Manoview | 127 ± 43 | 128 ± 41 | .74 |
| Anal pressure - Valsalva, new analysis | | | |
| Rectal pressure - rest, Manoview | 4 ± 7 | 32 ± 20 | <.0001 |
| Rectal pressure - rest, new analysis | 7 ± 6 | 33 ± 20 | <.0001 |
| Rectal pressure - evacuation, Manoview | 33 ± 20 | 66 ± 40 | <.0001 |
| Rectal pressure - evacuation, new | 33 ± 18 | 63 ± 34 | <.0001 |
| analysis | | | |
| Rectal pressure increment - evacuation, | 29 ± 19 | 34 ± 30 | .03 |
| Manoview | | | |
| Rectal pressure increment - evacuation, | 26 ± 17 | 30 ± 21 | .05 |
| new analysis | | | |
| Anal pressure - evacuation, Manoview | 73 ± 22 | 83 ± 25 | .0006 |
| Anal pressure - evacuation, new analysis | 65 ± 22 | 67 ± 21 | .44 |
| Change in anal pressures - evacuation, | −12 ± 31 | −13 ± 32 | .66 |
| Manoview | | | |
| Change in anal pressures - evacuation, | −2 ± 17 | −12 ± 19 | .0002 |
| new analysis | | | |
| Rectoanal gradient - evacuation, | −39 ± 25 | −17 ± 42 | <.0001 |
| Manoview | | | |
| Rectoanal gradient - evacuation, new | −32 ± 28 | −4 ± 39 | <.0001 |
| analysis | | | |
| First sensation | 45 ± 14 | 46 ± 17 | .71 |
| Desire to defecate | 70 ± 17 | 67 ± 20 | .51 |
| Urge | 117 ± 29 | 123 ± 46 | .42 |

TABLE 1B

| | Constipated (n = 136) | | |
|---|---|---|---|
| | Left | | |
| | lateral | Seated | p- |
| Parameter | (LL) | (UP) | value |
| Anal pressure - rest, Manoview | 90 ± 28 | 105 ± 34 | <.0001 |
| Anal pressure - rest, new analysis | 68 ± 26 | 79 ± 29 | <.0001 |
| Anal pressure - squeeze, Manoview | 183 ± 67 | 159 ± 68 | <.0001 |
| Anal pressure - squeeze, new analysis | | | |
| Anal pressure - Valsalva, Manoview | 125 ± 44 | 133 ± 48 | .004 |
| Anal pressure - Valsalva, new analysis | | | |
| Rectal pressure - rest, Manoview | 6 ± 8 | 28 ± 16 | <.0001 |
| Rectal pressure - rest, new analysis | 8 ± 11 | 30 ± 17 | <.0001 |
| Rectal pressure - evacuation, Manoview | 33 ± 28 | 62 ± 36 | <.0001 |
| Rectal pressure - evacuation, new | 32 ± 26 | 58 ± 29 | <.0001 |
| analysis | | | |
| Rectal pressure increment - evacuation, | 27 ± 26 | 33 ± 28 | .02 |
| Manoview | | | |
| Rectal pressure increment - evacuation, | 24 ± 22 | 28 ± 21 | .02 |
| new analysis | | | |
| Anal pressure - evacuation, Manoview | 83 ± 27 | 97 ± 34 | <.0001 |
| Anal pressure - evacuation, new analysis | 74 ± 28 | 81 ± 31 | .005 |
| Change in anal pressures - evacuation, | −4 ± 26 | −4 ± 29 | .60 |
| Manoview | | | |
| Change in anal pressures - evacuation, | 6 ± 18 | 2 ± 20 | .02 |
| new analysis | | | |
| Rectoanal gradient- evacuation, | −51 ± 33 | −35 ± 46 | <.0001 |
| Manoview | | | |
| Rectoanal gradient- evacuation, new | −42 ± 32 | −23 ± 41 | <.0001 |
| analysis | | | |
| First sensation | 49 ± 20 | 54 ± 23 | .02 |
| Desire to defecate | 84 ± 32 | 87 ± 34 | .45 |
| Urge | 123 ± 45 | 125 ± 46 | .83 |

TABLE 1C

| | p-value | p-value |
|---|---|---|
| | LL-Healthy | UP-Healthy |
| | vs | vs |
| Parameter | constipated | constipated |
| Anal pressure - rest, Manoview | .20 | .07 |
| Anal pressure - rest, new analysis | .80 | .85 |
| Anal pressure - squeeze, Manoview | .02 | .0001 |
| Anal pressure - squeeze, new analysis | | |
| Anal pressure - Valsalva, Manoview | .72 | .47 |
| Anal pressure - Valsalva, new analysis | | |
| Rectal pressure - rest, Manoview | .16 | .15 |
| Rectal pressure - rest, new analysis | .25 | .34 |
| Rectal pressure - evacuation, Manoview | .94 | .39 |
| Rectal pressure - evacuation, new analysis | .91 | .30 |
| Rectal pressure increment - evacuation, | .46 | .58 |
| Manoview | | |
| Rectal pressure increment - evacuation, | .58 | .43 |
| new analysis | | |
| Anal pressure - evacuation, Manoview | .002 | .001 |
| Anal pressure - evacuation, new analysis | .007 | .0002 |
| Change in anal pressures - evacuation, | .07 | .04 |
| Manoview | | |
| Change in anal pressures - evacuation, | .001 | <.0001 |
| new analysis | | |
| Rectoanal gradient- evacuation, Manoview | .006 | .004 |
| Rectoanal gradient- evacuation, new analysis | .03 | .001 |
| First sensation | .21 | .05 |
| Desire to defecate | .002 | .0001 |
| Urge | .39 | .90 |

Identification of Patterns with New Analysis

The new analysis classified rectoanal pressure profiles into four groups (i.e., minimal change, anal relaxation, paradoxical contraction, and transmission). In contrast to the Manoview™ analysis of all patients, the seated gradient was better than the left lateral gradient for discriminating between constipated patients with normal and abnormal BET.

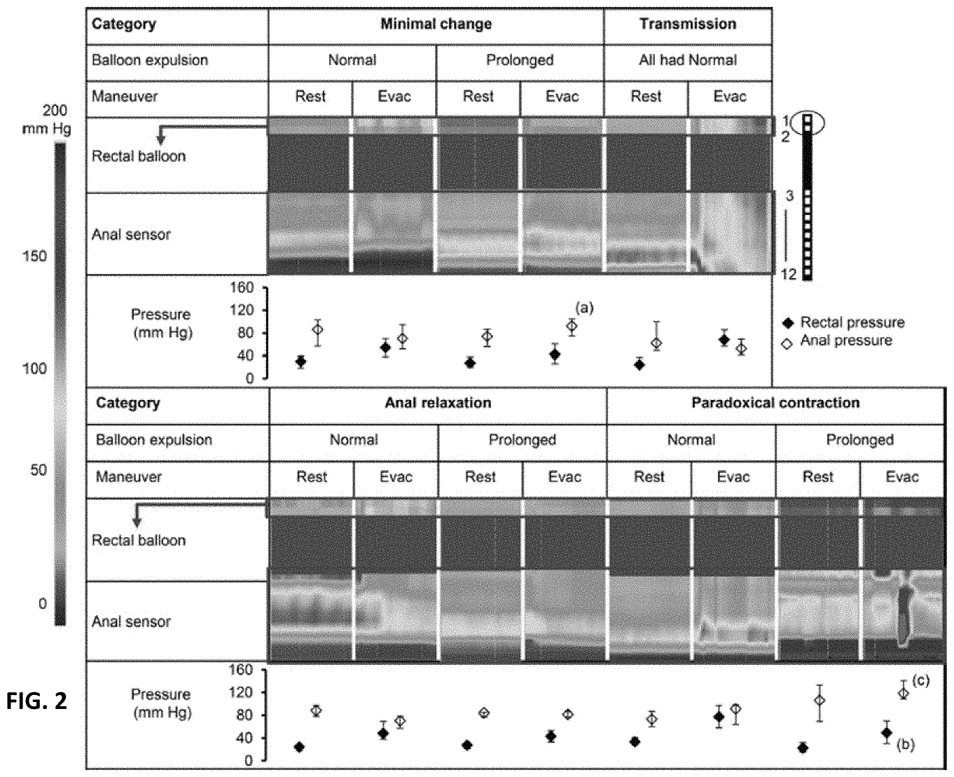

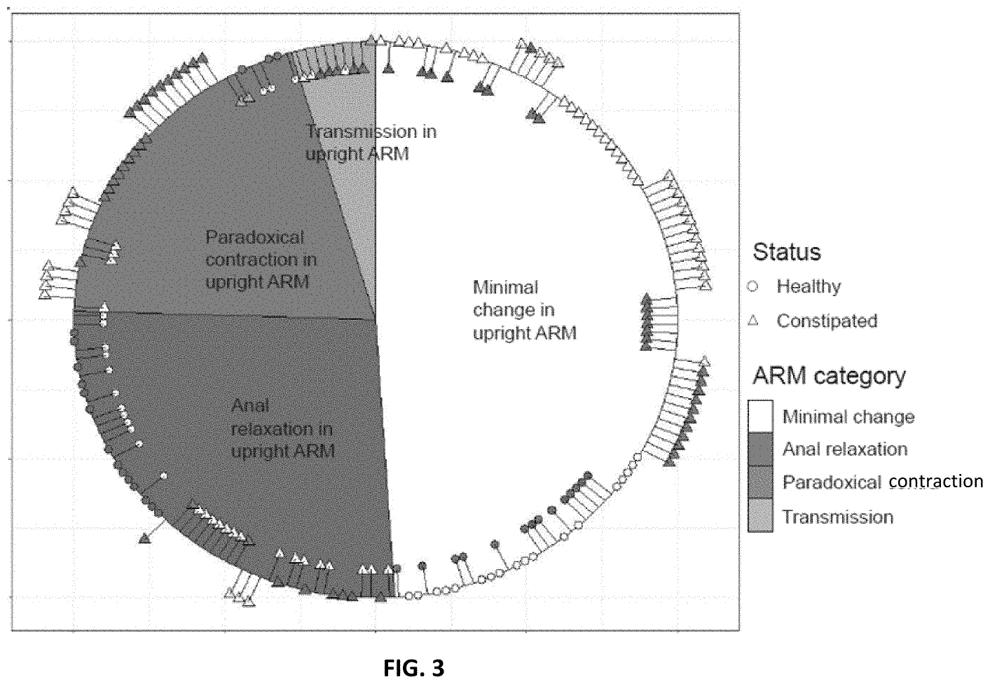

In the seated position, 9 of 136 constipated patients, all with a normal BET, had transmission (see FIGS. 2 and 3). The other patterns were anal relaxation (26 healthy women and 27 patients), paradoxical contraction (6 healthy and 34 constipated women), and minimal change (32 healthy and 66 constipated women). In the LL position, 3 of 136 constipated patients, who had a normal BET, satisfied the criteria for transmission with the new analysis (FIGS. 2 and 3). The remaining participants had anal relaxation (35 healthy women and 15 constipated patients), paradoxical contraction (12 healthy women and 46 patients), and minimal change (17 healthy and 72 constipated women).

Utility of Rectoanal Pressure Measurements in Both Positions for Discriminating Among Groups

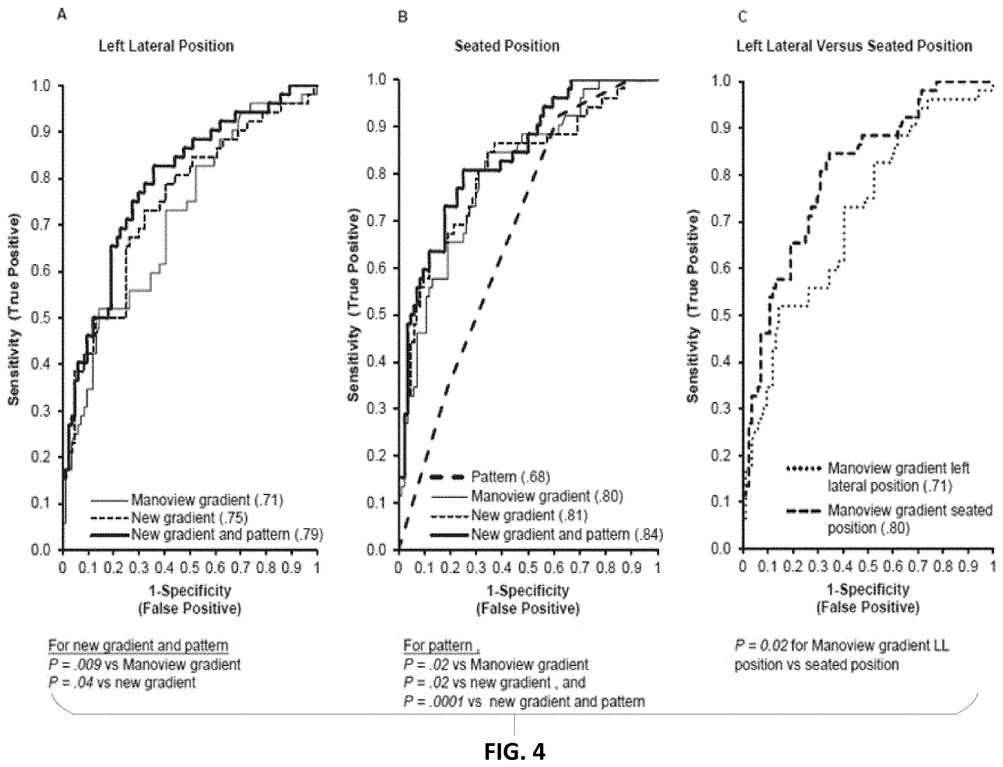

Table 3 and FIG. 4 depict the utility of rectoanal pressure parameters for discriminating between constipated patients with a normal and abnormal BET. The thresholds for these comparisons were derived from the distribution of values in healthy controls. For the rectal pressure increment and the rectoanal gradient, values lower than the 10^{th} percentile value in healthy controls was considered abnormal. For the anal pressure, values greater than 90^{th} percentile value in healthy controls was considered abnormal.

Among all parameters, the rectoanal gradient was the most useful for discriminating between healthy people and constipated patients with an abnormal BET and between constipated patients with a normal and an abnormal BET (Table 3). For comparing between constipated patients with normal and abnormal BET, the likelihood ratios were numerically greater, signifying greater diagnostic utility, with the new than the Manoview™ analysis (i.e., 6.2 versus 3.1 in the left lateral and 7.8 versus 4.8 in the seated position). The likelihood ratio is defined as the likelihood an abnormal parameter suggests abnormal balloon expulsion test over normal balloon expulsion test.

The position also affected the diagnostic utility of these tests. Some measures (e.g., rectal pressure increment) were greater in the seated than the left lateral position. Others (e.g. rectoanal gradient) were more useful when assessed in the seated than the left lateral position (i.e., 7.8 versus 6.2 for the rectoanal gradient evaluated with the new analysis. The AUC for the new rectoanal gradient (alone) for discriminating between constipated patients with normal and abnormal balloon expulsion tests was 0.75 in the left lateral and 0.81 in the seated positions (FIG. 4). The AUC for the integrated new analysis (i.e., rectoanal gradient and pattern) for discriminating between constipated patients with normal and abnormal balloon expulsion tests was 0.79 in the left lateral and 0.84 in the seated positions.

While Tables 2A and 2B compare the rectoanal gradient in left lateral and seated positions for all constipated patients with normal and abnormal (prolonged) BET, Tables 4A-4C compare the rectoanal gradient amongst the individual patterns (i.e., comparison of rectoanal pressures in constipated patients with normal and abnormal BET in LL and UP positions). Consistent with the definitions of these patterns, anal pressure declined and increased during evacuation respectively in participants with anal relaxation and paradoxical contraction. In all patterns, the rectoanal gradient was greater (i.e., less negative) in the seated than the left lateral position and likewise in constipated patients with normal than abnormal BET. The pattern was associated with the distribution of patients with normal and abnormal BET in the LL and separately the seated position. In the LL position, 60% with paradoxical contraction, 60% with minimal change, 93% with anal relaxation, and 100% with transmission had a normal BET. In the seated position, these proportions were respectively 66%, 55%, 85%, and 100%.

TABLE 2A

| | Left lateral position | | |
|---|---|---|---|
| | Consti- | Consti- | |
| | pated, | pated, | |
| | normal | abnormal | |
| | BET | BET | p- |
| Anorectal pressures, mm Hg | (n = 84) | (n = 52) | value |
| Recto-anal parameters | | | |
| Anal pressure - rest, Manoview | 85 ± 28 | 96 ± 27 | .03 |
| Anal pressure - rest, new analysis | 65 ± 26 | 73 ± 24 | .07 |
| Anal pressure - squeeze, Manoview | 194 ± 66 | 182 ± 67 | .31 |
| Anal pressure - squeeze, new analysis | | | |
| Anal pressure - Valsalva, Manoview | 119 ± 43 | 132 ± 42 | .08 |
| Anal pressure - Valsalva, new analysis | | | |
| Rectal pressure - rest, Manoview | 7 ± 9 | 5 ± 8 | .08 |
| Rectal pressure - rest, new analysis | 8 ± 12 | 8 ± 9 | .88 |
| Rectal pressure - evacuation, Manoview | 36 ± 27 | 27 ± 78 | 0.06 |
| Rectal pressure - evacuation, new | 36 ± 27 | 26 ± 23 | .02 |
| analysis | | | |
| Rectal pressure increment - evacuation, | 32 ± 27 | 20 ± 24 | 0.008 |
| Manoview | | | |
| Rectal pressure increment - evacuation, | 28 ± 23 | 18 ± 19 | .006 |
| new analysis | | | |
| Anal pressure - evacuation, Manoview | 78 ± 27 | 93 ± 26 | .002 |
| Anal pressure - evacuation, new analysis | 68 ± 27 | 84 ± 27 | .0007 |
| Change in anal pressures - evacuation, | −5 ± 31 | −1 ± 15 | 0.29 |
| Manoview | | | |
| Change in anal pressures - evacuation, | 3 ± 19 | 12 ± 16 | .007 |
| new analysis | | | |
| Rectoanal gradient - evacuation, | −42 ± 30 | −65 ± 33 | <.0001 |
| Manoview | | | |
| Rectoanal gradient - evacuation, | −32 ± 29 | −59 ± 32 | <.0001 |
| new analysis | | | |
| First sensation | 46 ± 20 | 52 ± 19 | .12 |
| Desire to defecate | 82 ± 32 | 88 ± 33 | .40 |
| Urge | 117 ± 43 | 132 ± 46 | .11 |

TABLE 2B

| | Seated position | | |
|---|---|---|---|
| | Consti- | Consti- | |
| | pated, | pated, | |
| | normal | abnormal | |
| | BET | BET | p- |
| Anorectal pressures, mm Hg | (n = 84) | (n = 52) | value |
| Recto-anal parameters | | | |
| Anal pressure - rest, Manoview | 98 ± 34 | 116 ± 31 | .002 |
| Anal pressure - rest, new analysis | 73 ± 27 | 89 ± 31 | .003 |
| Anal pressure - squeeze, Manoview | 189 ± 65 | 198 ± 59 | .37 |
| Anal pressure - squeeze, new analysis | | | |
| Anal pressure - Valsalva, Manoview | 123 ± 49 | 150 ± 42 | .001 |
| Anal pressure - Valsaiva, new analysis | | | |
| Rectal pressure - rest, Manoview | 27 ± 14 | 29 ± 17 | .43 |
| Rectal pressure - rest, new analysis | 31 ± 19 | 27 ± 13 | .12 |
| Rectal pressure - evacuation, Manoview | 67 ± 33 | 50 ± 30 | .003 |
| Rectal pressure - evacuation, new | 64 ± 30 | 48 ± 25 | .0009 |
| analysis | | | |
| Rectal pressure increment - evacuation, | 38 ± 29 | 24 ± 23 | .001 |
| Manoview | | | |
| Rectal pressure increment - evacuation, | 32 ± 21 | 21 ± 18 | .0008 |
| new analysis | | | |
| Anal pressure - evacuation, Manoview | 86 ± 29 | 115 ± 34 | <.0001 |
| Anal pressure - evacuation, new analysis | 70 ± 25 | 98 ± 33 | <.0001 |
| Change in anal pressures - evacuation, | −7 ± 35 | −1 ± 17 | .11 |
| Manoview | | | |
| Change in anal pressures - evacuation, | −3 ± 21 | 9 ± 17 | .0006 |
| new analysis | | | |
| Rectoanal gradient - evacuation, | −19 ± 35 | −64 ± 43 | <.0001 |
| Manoview | | | |
| Rectoanal gradient - evacuation, new | −7 ± 31 | −50 ± 40 | <.0001 |
| analysis | | | |
| First sensation | 51 ± 24 | 57 ± 22 | .14 |
| Desire to defecate | 82 ± 30 | 94 ± 37 | .07 |
| Urge | 116 ± 43 | 137 ± 53 | .03 |

TABLE 3

| | Healthy vs | | | | | |
|---|---|---|---|---|---|---|
| | constipated, | | | Constipated, normal | | |
| | abnormal BET | | | vs abnormal BET | | |
| | | | Like- | | | Like- |
| | Sen- | Spec- | lihood | Sen- | Spec- | lihood |
| Rectoanal parameter | sitivity | ificity | ratio | sitivity | ificity | ratio |
| Left lateral position | | | | | | |
| Rectal pressure increment- | 44 | 89 | 4.0 | 44 | 86 | 3.1 |
| evacuation, Manoview | | | | | | |
| analysis | | | | | | |
| Rectal pressure increment- | 23 | 89 | 2.1 | 23 | 95 | 4.8 |
| evacuation, new analysis | | | | | | |
| Anal pressure-evacuation, | 31 | 89 | 2.8 | 31 | 86 | 2.2 |
| Manoview analysis | | | | | | |
| Anal pressure-evacuation, | 38 | 89 | 3.5 | 38 | 88 | 3.2 |
| new analysis | | | | | | |
| Rectoanal gradient- | 52 | 89 | 4.7 | 52 | 83 | 3.1 |
| evacuation, Manoview | | | | | | |
| analysis | | | | | | |
| Rectoanal gradient- | 44 | 89 | 4.0 | 44 | 93 | 6.2 |
| evacuation, new analysis | | | | | | |
| Two or more abnormal | 60 | 86 | 4.2 | 60 | 73 | 2.2 |
| criteria-Manoview | | | | | | |
| analysis | | | | | | |
| Two or more abnormal | 50 | 81 | 2.7 | 50 | 75 | 2.0 |
| criteria-new analysis | | | | | | |
| Seated position | | | | | | |
| Rectal pressure increment- | 21 | 89 | 1.9 | 21 | 90 | 2.2 |
| evacuation, Manoview | | | | | | |
| analysis | | | | | | |
| Rectal pressure increment- | 21 | 89 | 1.9 | 21 | 89 | 2.0 |
| evacuation, new analysis | | | | | | |
| Anal pressure-evacuation, | 35 | 89 | 3.2 | 35 | 85 | 2.2 |
| Manoview analysis | | | | | | |
| Anal pressure-evacuation, | 58 | 89 | 5.3 | 58 | 81 | 3.0 |
| new analysis | | | | | | |
| Rectoanal gradient- | 52 | 89 | 4.7 | 52 | 89 | 4.8 |
| evacuation, Manoview | | | | | | |
| analysis | | | | | | |
| Rectoanal gradient- | 56 | 89 | 5.1 | 56 | 93 | 7.8 |
| evacuation, revised | | | | | | |
| analysis | | | | | | |
| Two or more abnormal | 65 | 83 | 3.8 | 65 | 77 | 2.9 |
| criteria-Manoview | | | | | | |
| analysis | | | | | | |
| Two or more abnormal | 71 | 81 | 3.8 | 71 | 69 | 2.3 |
| criteria-revised analysis | | | | | | |

TABLE 4A

| | | Rectal pressure increment | |
|---|---|---|---|
| Category | | Left lateral | Seated |
| Minimal | Constipated, | N = 43 | N = 36 |
| change | normal BET | | |
| | Constipated, | N = 29 | N = 30 |
| | abnormal BET | | |
| | Constipated, | 20 (12-29) | 24 (18-34) |
| | normal BET | | |
| | Constipated, | 8 (2-14) | 10 (6-21) |
| | abnormal BET | | |
| Anal | Constipated, | N = 14 | N = 23 |
| relaxation | normal BET | | |
| | Constipated, | N = 1 | N = 4 |
| | abnormal BET | | |
| | Constipated, | 19 (12-24) | 22 (17-32) |
| | normal BET | | |
| | Constipated, | | 16 (12-21) |
| | abnormal BET | | |
| Paradoxical | Constipated, | N = 24 | N = 16 |
| contraction | normal BET | | |
| | Constipated, | N = 22 | N = 18 |
| | abnormal BET | | |
| | Constipated, | 32 (14-65) | 45 (30-56) |
| | normal BET | | |
| | Constipated, | 21 (7-40) | 23 (12-38) |
| | abnormal BET | | |
| Transmission | Constipated, | N = 3 | N = 9 |
| | normal BET | | |
| | Constipated, | N = 0 | N = 0 |
| | abnormal BET | | |
| | Constipated, | 44 (26-59) | 72 (56-74) |
| | normal BET | | |
| | Constipated, | | |
| | abnormal BET | | |

TABLE 4B

| | | Change in anal pressure | |
|---|---|---|---|
| Category | | Left lateral | Seated |
| Minimal | Constipated, | N = 43 | N = 36 |
| change | normal BET | | |
| | Constipated, | N = 29 | N = 30 |
| | abnormal BET | | |
| | Constipated, | 3 (−4-6) | 0 (−7-14) |
| | normal BET | | |
| | Constipated, | 2 (−1-8) | 3 (−3-8) |
| | abnormal BET | | |
| Anal | Constipated, | N = 14 | N = 23 |
| relaxation | normal BET | | |
| | Constipated, | N = 1 | N = 4 |
| | abnormal BET | | |
| | Constipated, | −13 (−24-−3) | −17 (−22-−7) |
| | normal BET | | |
| | Constipated, | | −3 (−8-9) |
| | abnormal BET | | |
| Paradoxical | Constipated, | N = 24 | N = 16 |
| contraction | normal BET | | |
| | Constipated, | N = 22 | N = 18 |
| | abnormal BET | | |
| | Constipated, | 16 (10-24) | 12 (0-28) |
| | normal BET | | |
| | Constipated, | 19 (11-26) | 17 (8-20) |
| | abnormal BET | | |
| Transmission | Constipated, | N = 3 | N = 9 |
| | normal BET | | |
| | Constipated, | N = 0 | N = 0 |
| | abnormal BET | | |
| | Constipated, | 0 (−20-1) | −12 (−49-42) |
| | normal BET | | |
| | Constipated, | | |
| | abnormal BET | | |

TABLE 4C

| | | Rectoanal gradient | |
|---|---|---|---|
| Category | | Left lateral | Seated |
| Minimal | Constipated, | N = 43 | N = 36 |
| change | normal BET | | |
| | Constipated, | N = 29 | N = 30 |
| | abnormal BET | | |
| | Constipated, | −32 (−48-−3) | −6 (−33-11) |
| | normal BET | | |
| | Constipated, | −54 (−69-−38) | −49 (−68-−18) |
| | abnormal BET | | |
| Anal | Constipated, | N = 14 | N = 23 |
| relaxation | normal BET | | |
| | Constipated, | N = 1 | N = 4 |
| | abnormal BET | | |
| | Constipated, | −46 (−60-−37) | −15 (−32-12) |
| | normal BET | | |
| | Constipated, | | −37 (−45-−32) |
| | abnormal BET | | |
| Paradoxical | Constipated, | N = 24 | N = 16 |
| contraction | normal BET | | |
| | Constipated, | N = 22 | N = 18 |
| | abnormal BET | | |
| | Constipated, | −29 (−61-−19) | 1 (−16-14) |
| | normal BET | | |
| | Constipated, | −76 (−94-−58) | −57 (−104-−36) |
| | abnormal BET | | |
| Transmission | Constipated, | N = 3 | N = 9 |
| | normal BET | | |
| | Constipated, | N = 0 | N = 0 |
| | abnormal BET | | |
| | Constipated, | 9 (2-42) | 16 (12-39) |
| | normal BET | | |
| | Constipated, | | |
| | abnormal BET | | |

### Discussion

Prompted by the limited utility of HRM for diagnosing DD in constipated patients, this study modified the process for conducting (i.e., seated rather than the left lateral position) and analyzing anorectal HRM. The existing and new approaches for conducting (i.e., left lateral and seated positions) and analyzing HRM (i.e., Manoview™ and new method) for discriminating between healthy controls, constipated patients with normal and abnormal BET were evaluated.

There are four key observations from this study. First, rectal and anal pressures and the rectoanal gradient were greater in the seated than the left lateral position. Second, by contrast to the existing Manoview™ analysis, which solely relies upon summarizing rectoanal pressures during evacuation, the new method for analyzing HRM enables rectoanal pressure profiles to be classified into four patterns, which are variably associated with DD, hence useful for predicting the risk of DD in constipated patients. Third, with the Manoview™ analysis, the seated gradient was better than the left lateral gradient for discriminating between patients with normal and abnormal BET, however the AUC for the ROC curve was not different for seated vs left lateral gradients. By comparison, with the new analysis, the seated rectoanal gradient was numerically and statistically better than the left lateral gradient for discriminating between constipated patients with a normal and abnormal BET; the likelihood ratios were 6.2 (new analysis) versus 3.1 (Manoview™ program) in the left lateral position and 7.8 versus 4.8 (Manoview™ program) in the seated position.

Analysis of Evacuation with HRM

The existing (Manoview™) analysis identifies the most positive (or least negative) rectoanal gradient over 3 seconds during the evacuation maneuver, which typically lasts 20 seconds. While the rectal pressure is recorded by the same sensor throughout the 20 second maneuver, there are 3 limitations to the anal pressure used to calculate the gradient. Because the eSleeve options determines the highest pressure at any level of the anal canal sampled at 10 Hz, the gradient, is not the highest but actually the lowest gradient at that instant. In this context, “highest” refers to the highest value sustained for 3 seconds during the 20-second maneuver. Second, the location of the highest anal pressure may shift over 3 seconds. Third, the anal sensor used to compute the rectoanal gradient probably varies among patients. The new analysis overcomes these limitations by ensuring that the same sensors are used to record anal pressures in all patients for a longer duration (i.e., between 5-15 seconds after the onset of evacuation. The likelihood ratios for discriminating between constipated patients with normal and abnormal BET were greater when the rectoanal gradient was calculated by the revised approach than the existing software, which suggests the benefits of this approach.

Seated Manometry

Extending a previous study in healthy people, the rectal pressure, and to a lesser extent, anal pressure at rest and during evacuation were greater in the seated than the left lateral position. Among all participants, rectal pressures (i.e., on average by 30 mmHg) and to a lesser extent anal pressures (i.e., on average by 10 mmHg), were also higher in the seated than in the supine position. Hence, compared to the left lateral position, the rectoanal gradient was less negative in the seated than in the left lateral position. The rectoanal gradient discriminated between controls and patients with an abnormal BET in the seated but not in the left lateral position. Because patients with an abnormal BET probably have pelvic floor dysfunction, the area under these ROC curves, i.e., 0.80 in the seated and 0.71 in the left lateral position, is better than the corresponding value (i.e., 0.55) for discriminating between controls and patients with a normal BET in this study and 0.64, reported by Grossi et al for the comparison of controls and functional constipation. At a specificity of 80%, the rectoanal gradient was 73% sensitive for discriminating between controls and patients with an abnormal BET. In the Grossi study, type IV dyssynergia was 46% sensitive and 80% specific for discriminating between controls and FC patients.

HRM Patterns

Currently, rectoanal pressures during evacuation are only analyzed by computing the rectoanal gradient. While simple, this approach is inherently limited because it ignores the vast amount of information embedded in pressure topography plots of evacuation and the change from rest to evacuation. The new analysis provides a system that only uses the anal pressure change during evacuation to classify the rectoanal pressures into four categories (i.e., minimal change, anal relaxation, minimal change, paradoxical contraction, and transmission). The formulas for these calculations are simple and based of the raw data that can be extracted from the program. Among constipated patients, the proportion who had an abnormal BET in the seated position was 0% anal transmission, 15% for anal relaxation, 45% for minimal change, and 34% for paradoxical contraction. Thereafter, the rectoanal gradient, which was not used to classify pressure topography, was useful for discriminating between patients with normal and abnormal BET in all categories except for anal relaxation both during seated and LL manometry. This contrasts to the ROC analysis of the rectoanal gradient in the seated and left lateral positions analyzed by the Manoview™ program in all constipated patients. Transmission, an uncommon pattern, is characterized by increased rectal pressure that is transmitted to the anal canal, which implies that the anal canal is at least partly open. Indeed, all nine participants with transmission during seated position had a normal BET. In 57% of constipated patients, the rectoanal pressure profile during evacuation was different during LL and seated HRM, likely reflecting different patterns of abdomino-pelvic motion in these two positions.

Clinical Implications

Three observations suggest that the new analysis is preferable to the existing Manoview™ analysis for discriminating between constipated patients with normal and abnormal BET. First, for the rectoanal gradient the AUC for ROC curves derived from the individual patterns identified by the new analysis was greater than the corresponding Manoview™ analysis in all constipated patients. Second, with some patterns, a high proportion of patients, i.e., 100% with transmission and 80% with anal relaxation, which can only be identified with the new analysis, have normal balloon expulsion. Third, among these and other patterns, the rectoanal gradient is useful to discriminate among patients with normal and abnormal BET. Taken together, these findings support a new paradigm that, by comparison to current concepts, strongly suggests that HRM is useful for diagnosing, excluding, or prompting further tests to diagnose DD.

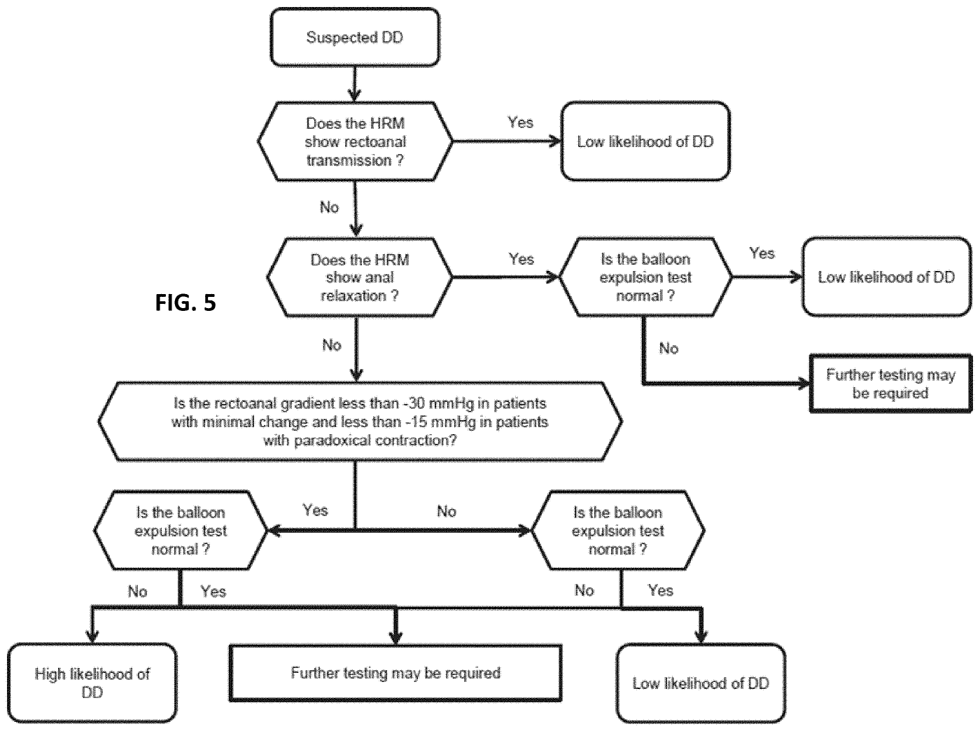

FIG. 5 provides an algorithm that incorporates these high resolution manometry findings and the balloon expulsion test to diagnose defecatory disorders. Among patients with transmission and AR who have an nBET, further testing is probably unnecessary unless warranted (eg, to assess for a large rectocele. Among patients with minimal change or paradoxical contraction, the value of the rectoanal gradient is useful for estimating the likelihood of a DD. When the gradient is greater (ie, less negative) than those thresholds and the BET is normal, the likelihood of a DD is probably low and additional tests seem unnecessary. When the gradient is lower than these thresholds and the BET is prolonged, the likelihood of a DD is probably high and additional tests seem unnecessary. Intermediate scenarios likely warrant further testing. By enhancing the utility of HRM for diagnosing DD, these findings will reduce the need for barium defecography, which entails radiation exposure and is not available at many centers.

Using a principal components analysis of rectoanal pressures measured with FIRM in a different cohort of 62 healthy and 295 constipated patients, we uncovered four key principal components or phenotypes in the cohort. Each principal component was defined by a weighted combination of several variables (e.g., anal resting pressure, rectal and anal pressures and the rectoanal gradient during evacuation). A score was computed for every PC in each patient. While that approach identified patterns or phenotypes (i.e. low rectal, high anal, and hybrid patterns), only approximately 50% of constipated patients with abnormal BET had abnormal values for one or more of four PCs. Hence, that approach, while useful for providing a pathogenesis-based classification of DD was less useful for distinguishing between healthy controls and patients with pelvic floor dysfunction. By contrast, the classification proposed in this study provides a means to classify patients into discrete categories—and thereafter to predict the likelihood of pelvic floor dysfunction.

Taken together, these observations demonstrate that HRM conducted in the upright position and analyzed by a new approach has a higher diagnostic utility for discriminating between constipated patients with normal and abnormal BET. Seated HRM and, to a lesser extent, LL HRM analyzed with a different approach are more useful than conventional LL HRM for discriminating between constipated patients with nBET and pBET.

While this specification contains many specific implementation details, these should not be construed as limitations on the scope of any invention or of what may be claimed, but rather as descriptions of features that may be specific to particular embodiments of particular inventions. Certain features that are described in this specification in the context of separate embodiments can also be implemented in combination in a single embodiment. Conversely, various features that are described in the context of a single embodiment can also be implemented in multiple embodiments separately or in any suitable subcombination. Moreover, although features may be described herein as acting in certain combinations and even initially claimed as such, one or more features from a claimed combination can in some cases be excised from the combination, and the claimed combination may be directed to a subcombination or variation of a subcombination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system modules and components in the embodiments described herein should not be understood as requiring such separation in all embodiments, and it should be understood that the described program components and systems can generally be integrated together in a single product or packaged into multiple products.

Particular embodiments of the subject matter have been described. Other embodiments are within the scope of the following claims. For example, the actions recited in the claims can be performed in a different order and still achieve desirable results. As one example, the processes depicted in the accompanying figures do not necessarily require the particular order shown, or sequential order, to achieve desirable results. In certain implementations, multitasking and parallel processing may be advantageous.

What is claimed is:

1. A method for assessing defecatory characteristics of a patient, the method comprising performing high-resolution anorectal manometry on the patient while the patient is in a seated position.

2. The method of claim 1, further comprising analyzing rectoanal pressure results from the high-resolution anorectal manometry, wherein the analyzing comprises classifying a rectoanal pressure profile of the patient from the high-resolution anorectal manometry into one of four groups: (i) minimal change, (ii) anal relaxation, (iii) paradoxical contraction, and (iv) transmission.

3. The method of claim 1, further comprising analyzing rectoanal pressure results from the high-resolution anorectal manometry, wherein the analyzing comprises determining a weighted combination of: (i) anal resting pressure, (ii) rectal and anal pressures and (iii) rectoanal gradient during evacuation.

4. The method of claim 1, wherein performing high-resolution anorectal manometry includes using a catheter comprising two rectal balloons with pressure sensors and ten additional pressure sensors spaced apart from each other along the catheter.

5. The method of claim 4, wherein at least seven pressure sensors of the ten additional pressure sensors are in the patient's anal canal while performing the high-resolution anorectal manometry.
